(19) United States
(12) Patent Application Publication
(10) Pub. No.: US 2015/0105649 A1
(43) Pub. Date: Apr. 16, 2015

(54) SUBJECT INFORMATION ACQUISITION APPARATUS

(71) Applicant: CANON KABUSHIKI KAISHA, Tokyo (JP)

(72) Inventors: Koichi Suzuki, Kodaira-shi (JP); Shigeru Ichihara, Tokyo (JP)

(21) Appl. No.: 14/391,688

(22) PCT Filed: Mar. 13, 2013
(86) PCT No.: PCT/JP2013/001646

§ 371 Date: Oct. 9, 2014

(30) Foreign Application Priority Data
Apr. 13, 2012 (JP) 2012-092274

(51) Int. Cl. A61B 5/00

(52) U.S. Cl. CPC A61B 5/0095; A61B 5/741
USPC 600/407

(57) ABSTRACT

It is possible to perform light emitting control to a subject taking into account measurement conditions (size and a pressing state of the subject).

A subject information acquisition apparatus includes a pressure unit that presses the subject, a light emitting unit that emits light to the subject through the pressure unit, a distance information acquisition unit that acquires information related to a distance between the pressure unit and the light emitting unit, and a control unit that controls an operation of the light emitting unit on the basis of the information related to the distance acquired by the distance information acquisition unit.

### TECHNICAL FIELD

The present invention relates to a subject information acquisition apparatus such as a photoacoustic apparatus.

### BACKGROUND ART

Conventionally, many photoacoustic apparatuses, which irradiate a subject with pulsed light, receive photoacoustic waves generated from inside the subject by a probe, and make an image of the shape and function in the subject, are studied in the medical field. Among them, a photoacoustic apparatus including a mechanism that presses and holds the subject in order to cause light to reach a deep portion of the subject is proposed. In a photoacoustic apparatus described in PTL 1, a practitioner sandwiches and fixes the subject between two pressure plates and then irradiates the subject with pulsed light emitted from a projector through a first pressure plate. The photoacoustic apparatus has a structure in which a probe receives photoacoustic waves generated from the subject through a second pressure plate.

Generally, when applying a photoacoustic apparatus to a living organism, to avoid damaging the living organism, it is necessary to limit the irradiation density to smaller than a predetermined threshold value (PTL 2). This value is called MPE (maximum permissible exposure).

### CITATION LIST

### Patent Literature

PTL 1: Japanese Patent Laid-Open No. 2010-17427

PTL 2: Japanese Patent Laid-Open No. 2010-147940

### SUMMARY OF INVENTION

### Technical Problem

The irradiation density of light varies depending on the distance from a projector. The distance between the projector and the subject varies depending on the size of the subject and the state of the pressure. Therefore, every time the subject (examinee) and the state of the pressure of the subject change, that is, every time the measurement is performed, the irradiation density may change. Thus, it is necessary to control the measurement so that the irradiation density does not exceed the MPE for each measurement.

### Solution to Problem

The present invention provides a subject information acquisition apparatus for acquiring characteristic information of a subject by receiving a photoacoustic wave generated by irradiating the subject with light. The subject information acquisition apparatus includes a pressure unit that presses the subject, a light emitting unit that emits light to the subject through the pressure unit, a distance information acquisition unit that acquires information related to a distance between the pressure unit and the light emitting unit, and a control unit that controls an operation of the light emitting unit on the basis of the information related to the distance acquired by the distance information acquisition unit.

Also, the present invention provides a method for acquiring characteristic information of a subject by receiving a photoacoustic wave generated when a light emitting unit emits light to the subject pressed by a pressure unit through the pressure unit. The method includes a step of pressing the subject by the pressure unit, a step of acquiring information related to a distance between the pressure unit and the light emitting unit, and a step of controlling an operation of the light emitting unit on the basis of the acquired information related to the distance.

### Advantageous Effects of Invention

According to the subject information acquisition apparatus or an acquisition method of characteristic information of the subject of the present invention, it is possible to control the irradiation density distribution of the light irradiated to the subject so that the irradiation density distribution does not exceed a predetermined value every time the characteristic information of the subject is acquired (measured).

Further features of the present invention will become apparent from the following description of exemplary embodiments with reference to the attached drawings.

### BRIEF DESCRIPTION OF DRAWINGS

FIG. 1 is a block configuration diagram according to a first embodiment of the present invention.

FIG. 2 is an operation flowchart according to the first embodiment of the present invention.

FIG. 3A is a diagram showing a positional relationship of components near a subject according to the first embodiment of the present invention.

FIG. 3B is a diagram showing a positional relationship of components near a subject according to the first embodiment of the present invention.

FIG. 4A is a configuration diagram of a projector according to the first embodiment of the present invention.

FIG. 4B is a diagram showing optical characteristics of the projector according to the first embodiment of the present invention.

FIG. 5A is a configuration diagram of a projector according to a modified example of the first embodiment of the present invention.

FIG. 5B is a diagram showing optical characteristics of the projector according to the modified example of the first embodiment of the present invention.

FIG. 6 is an operation flowchart according to a second embodiment of the present invention.

FIG. 7A is a diagram showing a relationship between an input energy and a distance according to the second embodiment of the present invention.

FIG. 7B is a diagram showing a relationship between an input energy and a distance according to the second embodiment of the present invention.

### DESCRIPTION OF EMBODIMENTS

### First Embodiment

A first embodiment of the present invention measures a distance between a pressure plate which is a pressure unit and a projector which configures a light emitting unit, and if the distance is out of a range previously stored in a memory in a subject information acquisition apparatus, emits no pulsed light (control of operation of the light emitting unit) and prompts a user to further press the subject. In the description below, the distance between the projector and the pressure plate is called a projection distance and the range stored in the memory is called an irradiation possible range. Hereinafter, the first embodiment of the present invention will be described in detail with reference to the drawings.

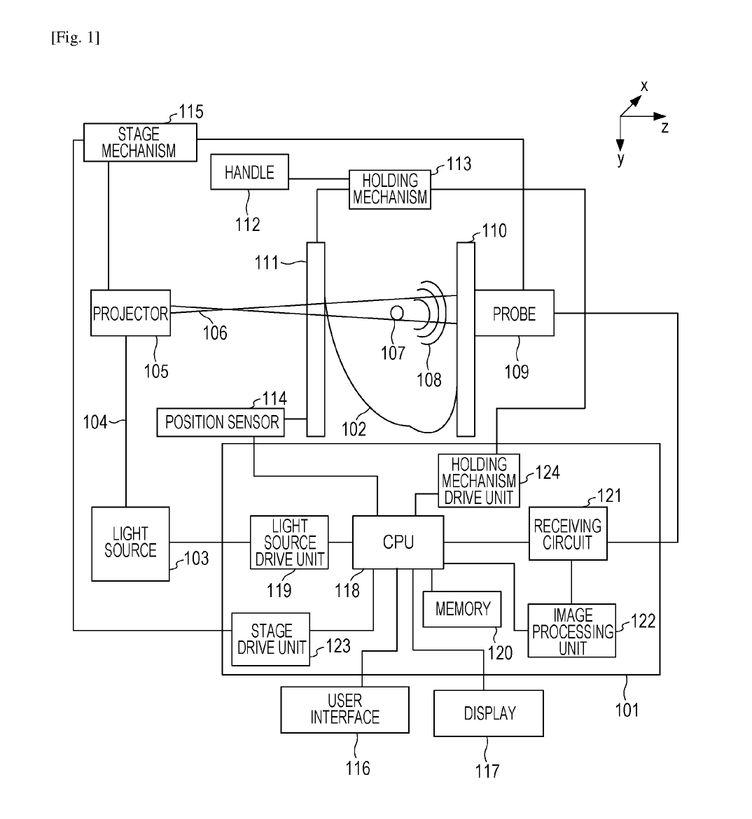

FIG. 1 is a block configuration diagram showing a photoacoustic apparatus which is a subject information acquisition apparatus according to an embodiment of the present invention. The photoacoustic apparatus which is the subject information acquisition apparatus of the present embodiment includes at least a pressure unit, a light emitting unit, a distance information acquisition unit, and a control unit that controls an operation of the light emitting unit. In the present embodiment shown in FIG. 1, the pressure unit includes a pair of pressure plates 110 and 111 which face each other and press a subject 102 and the light emitting unit that emits light to the subject 102 through the pressure unit includes a light source 103 and a projector 105. The distance information acquisition unit that acquires information related to the distance between the pressure unit and the light emitting unit includes a position sensor 114 and the control unit that controls the operation of the light emitting unit on the basis of the information related to the distance acquired by the distance information acquisition unit includes a light source drive unit 119 and a CPU 118.

As described above, the irradiation density of the light emitted from the light emitting unit varies depending on the distance from the light emitting unit (moving distance of emitted light). In the embodiments of the present invention, since the control unit controls the operation of the light emitting unit on the basis of the information related to the distance acquired by the distance information acquisition unit, even when the size of the subject or the pressed state of the subject changes, it is possible to control the irradiation density of the light emitted to the subject so that the irradiation density does not exceeds a predetermined value. More specifically, when the subject is replaced or the pressed state of the same subject changes, the positional relationship between the light emitting unit and the subject changes. As the positional relationship between the light emitting unit and the subject changes, also the position of the pressure unit changes. Therefore, it is possible to control the irradiation density of the light emitted to the subject so that the irradiation density does not exceeds a predetermined value by controlling the operation of the light emitting unit on the basis of the information related to the distance between the pressure unit and the light emitting unit. The position information of the pressure unit (information necessary to acquire the distance from the light emitting unit) is easier to be acquired than the position information of the subject. Therefore, it is desirable to control the light emitting unit on the basis of this information as a control of the subject information acquisition apparatus.

As a preferred embodiment, the apparatus configuration shown in FIG. 1 includes a memory 120, a receiving circuit 121, an image processing unit 122, a stage drive unit 123, and a holding mechanism drive unit 124 which are included, along with the CPU 118 and the light source drive unit 119 which are included in the control unit, in a controller 101 that controls an operation of the entire photoacoustic apparatus. Further, the apparatus configuration includes a light path 104 that connects the light source 103 with the projector 105, a probe 109, a handle 112, a holding mechanism 113, a stage mechanism 115, a user interface 116, and a display 117. Hereinafter, the apparatus configuration including the above components will be described in detail.

Reference numeral 101 denotes a controller that controls an operation of the entire photoacoustic apparatus. Reference numeral 102 denotes a subject which is a part of the body of an examinee Here, a breast will be described as an example the subject. Reference numeral 103 denotes a pulsed laser light source which is formed by a YAG laser, a titanium-sapphire laser, or the like. The pulsed laser light source includes a flash lamp and a Q switch as a means for exciting a laser medium inside the pulsed laser light source and the light emitting timing can be controlled from the outside. The pulsed laser light source includes an interface for setting an input energy and the energy of the pulsed light can be controlled from the outside. Reference numeral 104 denotes a light path for guiding the pulsed light to near the subject and the light path 104 is formed by a bundle fiber in which a large number of optical fibers are bundled. Reference numeral 105 denotes a projector that irradiates a measurement portion of the subject with the pulsed light. The projector 105 includes an optical system that magnifies light emitted from the bundle fiber 104 at a predetermined magnification and adjusts the density of irradiation light and an irradiation region. Reference numeral 106 denotes the pulsed light irradiated from the projector 105 to the subject 102. The direction of the pulsed light 106 is defined as a Z axis. A horizontal direction on a plane perpendicular to the Z axis is defined as an X axis and a vertical direction on the plane is defined as a Y axis. A position of the light emitting end of the projector 105 is defined as the origin of the Z axis. The details of the position of the light emitting end will be described later. Reference numeral 107 denotes a region showing a large optical absorption. An example of the region is a newborn blood vessel caused by a breast cancer. When the region 107 is irradiated with the pulsed light, a photoacoustic wave 108 is generated by a photoacoustic effect. Reference numeral 109 denotes a probe which includes a transducer for receiving the photoacoustic wave 108 inside the probe. The transducer is formed by arranging ultrasonic sensor elements such as PZT and CMUT in an array and the photoacoustic wave 108 is converted into an electrical signal. The electrical signal is called a photoacoustic signal. Reference numerals 110 and 111 are a pair of plates for pressing and fixing the subject 102. The plate 110 which is one of the pair of plates is located between the subject 102 and the probe 109 and fixed to be in contact with the probe 109. The plate 110 is formed of a material whose acoustic impedance is near that of the subject 102 and the plate 110 can efficiently transmit the photoacoustic wave 108 to the probe 109. If bubbles are mixed between the plate 110 and the subject 102 and between the plate 110 and the probe 109, the photoacoustic wave 108 reflects on the interface of the bubbles, so that the photoacoustic wave 108 does not reach the probe 109. Therefore, a liquid whose impedance is near that of the subject is inserted between the plate 110 and the subject 102 and between the plate 110 and the probe 109 as a matching agent. Castor oil and the like are used as the matching agent. When a user (practitioner) presses and fixes the subject 102, the user closely contacts the subject 102 with the plate 110 so that bubbles are not inserted. The plate 111 which is the other one of the pair of plates can move in the Z direction between the projector 105 and the subject 102. Thereby, it is possible to press and fix the subject 102 in various thicknesses according to the size of the subject 102. The plate 111 is formed of a highly light transmissive material and can efficiently irradiate the subject 102 with the pulsed light 106. Reference numeral 112 denotes an operation handle for the user (practitioner) to move the plate 111 and reference numeral 113 denotes a holding mechanism that transmits force of the operation handle to the pressure plate. When the handle is rotated, the plate 111 moves in the Z axis direction. The holding mechanism 113 includes a brake for fixing the position of the plate inside the holding mechanism and fixes the position or the plate 111 on the basis of an instruction of the user so that the plate 111 is not moved by reaction force from the subject 102. The user (practitioner) opens a door not shown in FIG. 1 and manually operates the subject 102, and then fixes the plate 111, closes the door, and starts capturing an image of the subject. Although the subject 102 is irradiated with the pulsed light, the light is blocked by the door, so that scattered light is prevented from hitting the user. Reference numeral 114 denotes a sensor for measuring the position of the plate 111. The sensor 114 is formed by, for example, a potentiometer and a wire connected to the plate 111. When the wire is pulled out by the movement of the plate 111, the resistance value of the potentiometer changes, so that it is possible to measure the position of the plate 111. The distance between the surface of the plate 111 facing the subject 102 and the light emitting end of the projector 105 is the projection distance. Reference numeral 115 denotes a mechanism that moves the probe 109 and the projector 105 in the XY direction. The mechanism 115 includes a motor, an XY stage, an encoder, and the like. By the mechanism 115, it is possible to scan the probe 109 and the projector 105 along the subject 102 and acquire a wide range of photoacoustic signals of the subject 102. Reference numeral 116 denotes a user interface for a user to set operating conditions of the photoacoustic apparatus and input operation instructions of the photoacoustic apparatus. The user interface 116 includes a keyboard, a mouse, button switches, and the like. The operating conditions include the measurement range of the subject 102, the measurement time of the photoacoustic signal, and the like. The operation instructions include a start of capturing an image of the subject, a stop of capturing an image of the subject, and the like. Reference numeral 117 denotes a display which is an example of a presentation unit. The display 117 displays a diagnostic image to the user and notifies the user of the state of the photoacoustic apparatus. Reference numeral 118 denotes a CPU that controls operations of the entire photoacoustic apparatus. The CPU 118 is formed by an embedded microcomputer and software. The CPU 118 receives an instruction from the user through the user interface 116 and operates the apparatus according to the instruction. Reference numeral 119 denotes a drive circuit of the light source 103. The drive circuit 119 turns on the flash lamp at a constant period, turns on the Q switch after accumulating excitation energy in the laser medium, and causes a pulsed light having high energy called a giant pulse to be outputted. The drive circuit 119 communicates with the light source 103 and sets an input energy to the flash lamp. It is possible to control the light quantity emitted from the light source by the amount of the input energy. Reference numeral 120 denotes a memory that stores content set by the user (for example, data of the irradiation possible range described later), acquired photoacoustic signals, diagnostic images, and the like.

The range in which the subject 102 is located is between the plate 110 and the plate 111. In the entire range, a range of the projection distance where the irradiation density is smaller than or equal to the MPE is the irradiation possible range, which is stored in the memory 120 in advance. The range where the irradiation density is smaller than or equal to the MPE will be described below.

The irradiation density of the light emitted from the projector is uneven and varies depending on the distance from the projector, so that the irradiation density is a value depending on three coordinates x, y, and z. The irradiation density is defined as P(x, y, z). The maximum value of the irradiation density in the XY plane is defined as Pmax(z). When the Z coordinate of the plate 110 is Z110 and the Z coordinate of the plate 111 is Z111, Z111 is equal to the projection distance. In a range of Z111<=z<=Z110, a range in which the Pmax(z) is smaller than or equal to the MPE is the irradiation possible range.

The CPU 118 emits the pulsed light from the light emitting unit to the subject 102 when the projection distance is within the irradiation possible range. The projection distance is obtained from a difference between the distance corresponding to an output from the position sensor 114 and the distance between the position sensor 114 and the light emitting end of the projector 105. Reference numeral 121 denotes a circuit that receives the photoacoustic signal from the probe 109. The circuit 121 includes a preamplifier, an A/D converter, a receiving memory, and an FPGA. The photoacoustic signal is amplified by the preamplifier, converted into a digital value by the A/D converter, and inputted into the FPGA. FPGA performs signal processing such as noise reduction processing and phase rectifying addition. The photoacoustic signal on which the signal processing is performed by the receiving circuit is stored in the memory 120. The data stored in the memory 120 is called photoacoustic signal data. Reference numeral 122 denotes a signal processing circuit which performs image reconstruction processing from the photoacoustic signal data, generates an image indicating an absorption coefficient distribution of the pulsed light in the subject 102, and displays the image on the display 117. In this image reconstruction processing, the range in which the subject 102 is located is obtained from the position of the plate 111 measured by the position sensor 114 and an image in the range is generated. A range in which Z coordinates are located between the plate 111 and the plate 110 is the range in which the subject 102 is located. Thereby, the calculation time can be smaller than that when generating an image of a region including the entire movable range of the plate 111. Reference numeral 123 denotes a circuit that controls the stage mechanism 115. The circuit 123 includes a 4-axis motor driver circuit. On the basis of the instruction from the CPU 118, the projector 105 and the probe 109 are scanned in the X and Y axis directions. Reference numeral 124 denotes a circuit that controls the brake of the holding mechanism. The circuit 124 can control the brake to switch fixed and movable of the plate 111 on the basis of the instruction inputted by the user through the user interface 116.

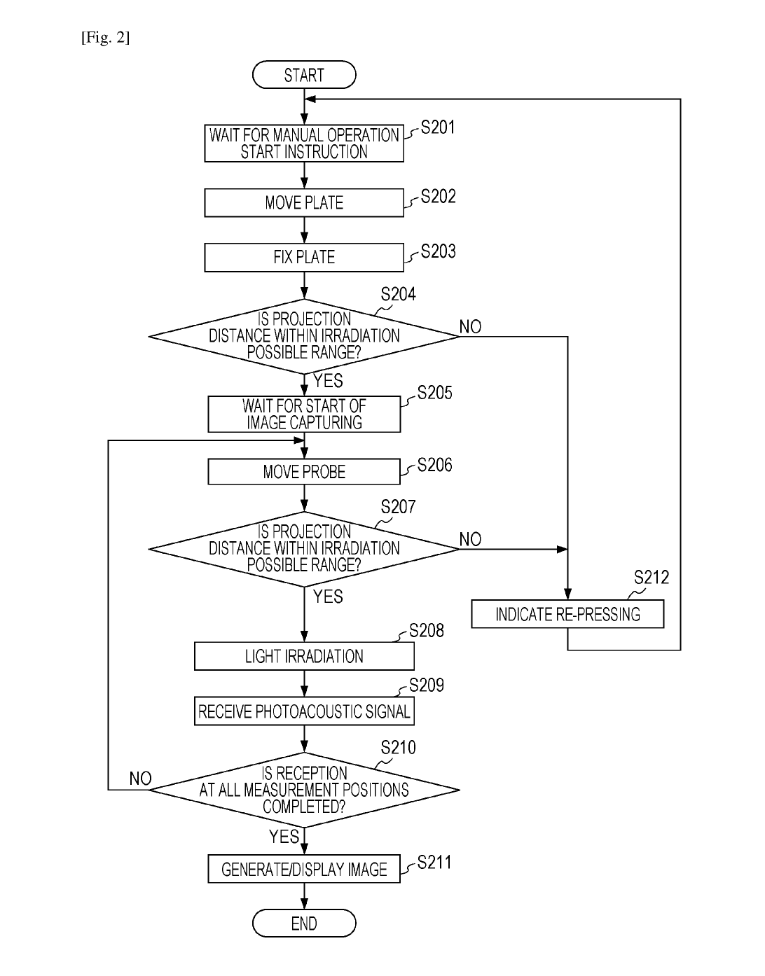

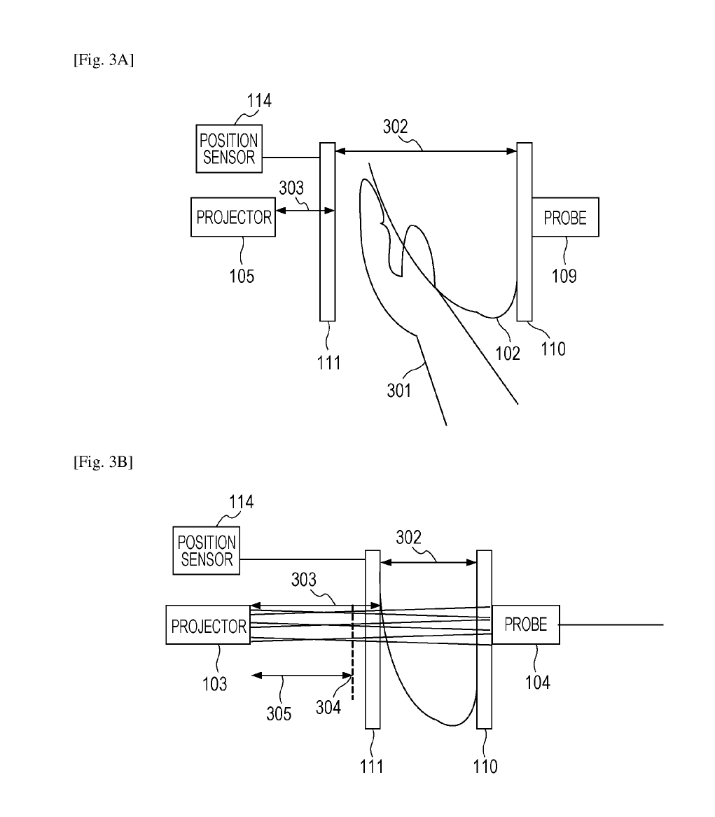

Next, an operation procedure of the photoacoustic apparatus will be described with reference to a flowchart in FIG. 2 and diagrams in FIGS. 3A and 3B. Step S201 in FIG. 2 shows a state in which the photoacoustic apparatus waits for starting pressure of the subject 102 by an operation of the handle 112 by a user. Steps S202 and S203 are a process for pressing the subject by the plate 111 which is a pressure unit. First, the user moves the handle 112, so that the plate 111 is moved in the Z axis direction through the holding mechanism 113. FIG. 3A shows a situation around the subject at this time. In FIG. 3A, the user closely attaches the subject 102 to the plate 110 by a hand 301 so that bubbles are not inserted between the plate 110 and the subject 102. To do so, first, the plate 111 is moved leftward (in the minus direction of the Z axis in FIG. 1) and the subject 102 and the hand 301 are inserted between the plate 110 and the plate 111. Subsequently, the subject 102 is stretched downward (in the plus direction of the Y axis in FIG. 1) by the hand 301 and pressed against the plate 110, the plate 111 is moved rightward (in the plus direction of the Z axis in FIG. 1) while the hand 301 is pulled out downward, and the subject 102 is pressed until the thickness of the subject 102 becomes a thickness in which the pulsed light reaches the inside of the subject 102. Then, in step S203, the position of the plate 111 is fixed on the basis of an instruction from the user and the pressure fixation of the subject is completed. The next step S204 is a process for acquiring information related to the distance between the plate 111 which is the pressure unit and the light emitting unit and a process for controlling the operation of the light emitting unit on the basis of the acquired information related to the distance. The CPU 118 reads the position of the plate 111 from the position sensor 114 and determines whether or not a projection distance 303 is within the irradiation possible range stored in the memory 120. As a result of the determination, if the projection distance 303 is out of the irradiation possible range, it is determined that the light irradiation is prohibited (impossible) and the CPU 118 proceeds to step S212. In step S212, the CPU 118 displays a message on the display 117 and prompts the user to further press the subject (in other words, prompts the user to increase the distance between the light emitting unit and the plate 111 which is the pressure unit) and the CPU 118 returns to step S201. Accordingly, the user presses the subject 102 again. On the other hand, as a result of the determination in step S204, if the projection distance 303 is within the irradiation possible range, it is determined that the light irradiation is possible and the CPU 118 proceeds to step S205. In step S205, it is waited that the user sets an image capturing condition such as an acquisition range of the photoacoustic signal and the number of times when the photoacoustic signal is acquired at one measurement point. When a plurality of photoacoustic signals are acquired at one measurement point and the arithmetic average of the photoacoustic signals is calculated, it is possible to improve the S/N ratio of the photoacoustic signal. When the user inputs an instruction of starting acquisition of the photoacoustic signal, the CPU 118 proceeds to step S206. In step S206, the CPU 118 issues an instruction to the stage drive unit 123, moves the motor of the stage mechanism 115, and moves the probe 109 and the projector 105 to the front of the measurement position of the subject 102.

Subsequently, in step S207, the same determination as that in step S204 is performed. As a result of the determination, if the projection distance 303 is out of the irradiation possible range, it is determined that the light irradiation is impossible and the CPU 118 proceeds to step S212. On the other hand, as a result of the determination, if the projection distance 303 is within the irradiation possible range, it is determined that the light irradiation is possible and the CPU 118 proceeds to step S208. In this way, the determination is performed immediately before the light irradiation, so that even when the position of the plate 111 is changed, the change can be detected and the irradiation can be cancelled.

Subsequently, in step S208, the CPU 118 issues an instruction to the light source drive unit 119 to cause the light source 103 to emit the pulsed light. FIG. 3B shows a situation around the subject at this time.

FIG. 3B shows paths of light from the upper end of the projector, light from the middle of the projector, and light from the lower end of the projector. The light from the projector 105 forms an image at a certain position and thereafter as the light approaches the subject, the light gradually widens. A position at which the light from the projector 105 sufficiently widens and the irradiation density is locally lower than the MPE is denoted by reference numeral 304. A range of the projection distance greater than or equal to the distance 305 from the projector 105 to the position 304 is the irradiation possible range. As shown in FIGS. 3A and 3B, when the user operates manually, the plate 111 is moved near the projector 105, the projection distance is set to out of the irradiation possible range, and space to insert a hand is ensured. On the other hand, when the light is emitted, the plate 111 is moved away from the projector 105 and the projection distance is set to within the irradiation possible range, so that the irradiation density of the subject 102 is smaller than or equal to the MPE.

In this way, the subject pressed by the pressure unit is irradiated with light by the light emitting unit through the plate 111 which is the pressure unit.

Subsequently, in step S209, the receiving circuit 121 receives the photoacoustic signal, performs signal processing such as amplification, A/D conversion, and noise reduction on the photoacoustic signal, and thereafter stores the photoacoustic signal in the memory 120. Then, the CPU 118 proceeds to step S210. In step S203, the CPU 108 determines whether or not the photoacoustic signal has been received at all measurement positions in a range specified by the user. If the photoacoustic signal has not been received at all the measurement positions, the CPU 118 returns to step S204 and moves the projector 105 and the probe 109 to the next measurement position. If the photoacoustic signal has been received at all the measurement positions, the CPU 118 proceeds to step S211. In step S211, the image processing circuit 122 performs image processing such as image reconstruction processing and scan conversion processing based on the photoacoustic signals stored in the memory 120, displays a photoacoustic image on the display 117, and completes the process. In this way, the photoacoustic waves generated by emitting light by the light emitting unit are received, so that the characteristic information of the subject is acquired and the characteristic information is displayed.

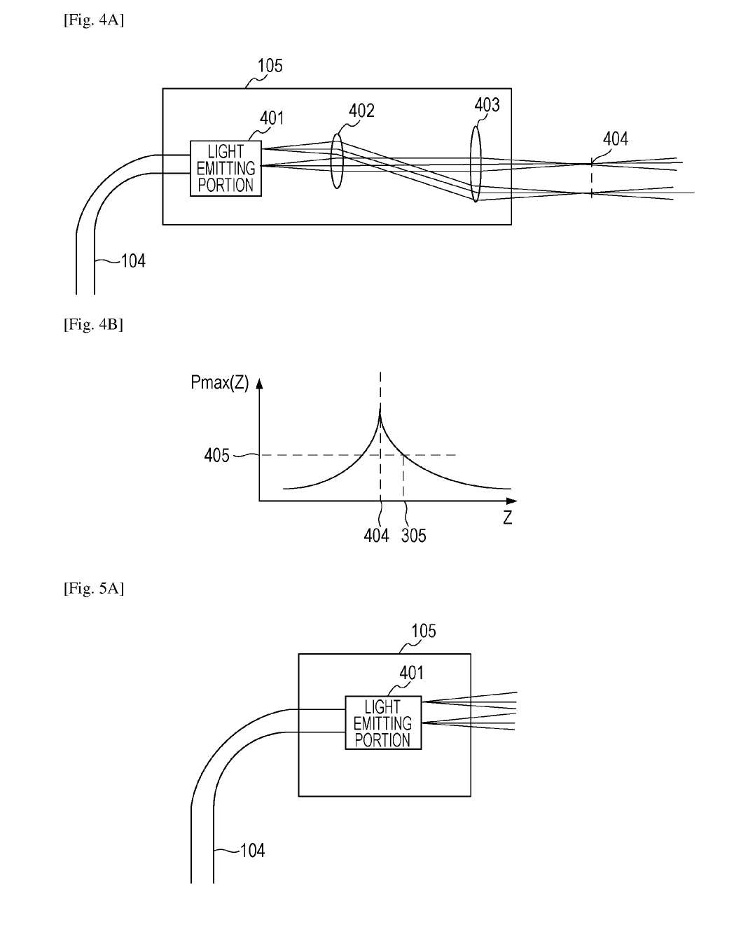

FIG. 4A shows an internal configuration of the projector 105. An optical system (hereinafter referred to as an “optical unit”) in the projector enlarges light emitted from a plurality of optical fibers by a lens system and causes the light to travel in parallel with an optical fiber light emitting portion. The light travels in parallel with the optical fiber light emitting portion, so that variation of the size of the irradiation region along the Z axis direction in the subject 102 is reduced. Reference numeral 401 denotes a light emitting portion of the optical fiber 104. Reference numerals 402 and 403 denote an optical unit including convex lenses and the optical unit is arranged to be a both sides telecentric optical system. Light from the fiber light emitting portion 401 is formed by overlapping light from a large number of bundle fibers and includes a locally high irradiation density portion and a locally low irradiation density portion. At a position where a value Pmax of the high irradiation density portion hits the subject, it is necessary to reduce the irradiation density to be smaller than the MPE. The light from the fiber light emitting portion 401 is enlarged through the lenses 402 and 403 and forms an image at the position 404, so that the peak Pmax of the irradiation density increases. As the light travels away from the image forming position 404, the image blurs, so that the peak Pmax of the irradiation density decreases. FIG. 4B shows the characteristics of the peak value Pmax of the irradiation density when the horizontal axis represents the Z axis and the vertical axis represents the peak value Pmax. The origin of the Z axis is the position of the lens 403 which is the light emitting end of the light emitting unit. Reference numeral 405 denotes the MPE corresponding to the wavelength and the frequency of the pulsed light from the light source 103. A Z coordinate which is farther from the projector 105 than the image forming position 404 and at which the irradiation density is equal to the MPE 405 is denoted by 305. In a range in which the Z coordinate is greater than or equal to the Z coordinate 305, the irradiation density is smaller than or equal to the MPE, so that the irradiation possible range is a range in which the projection distance is greater than or equal to the Z coordinate 305. In this way, the irradiation possible range is set so that the peak value of the irradiation density does not exceed the MPE. It is assumed that the irradiation possible range is obtained in advance from the characteristics of the optical system as shown in FIG. 4B and the irradiation possible range is stored in the memory 120.

In the present embodiment, when the projection distance is out of the irradiation possible range, a message is displayed on the display to prompt the user to press the subject again (in other words, prompt the user to increase the distance between the light emitting unit and the plate 111 which is the pressure unit). However, the notification method to the user is not limited to this. For example, a voice generation unit (sound source) is separately provided as a presentation unit and when the projection distance is out of the irradiation possible range, it is possible to notify the user accordingly by voice.

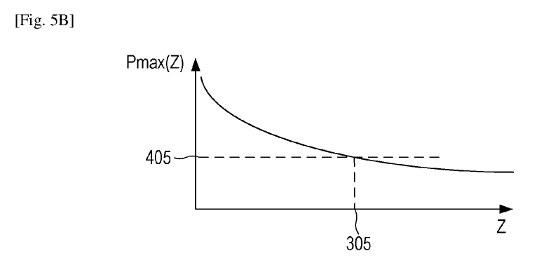

In the present embodiment, as the optical unit in the projector 105, an example of the both sides telecentric optical system is described. However, another optical system may be used. For example, a case in which the light from the light emitting portion 401 of the optical fiber is directly irradiated to the subject 102 will be described with reference to FIGS. 5A and 5B. In the case of FIG. 5A, the irradiation density decreases according to a widening angle of the optical fiber. FIG. 5B shows the characteristics of the peak value Pmax of the irradiation density when the horizontal axis represents the Z axis and the vertical axis represents the peak value Pmax. The origin of the Z axis is the position of the light emitting portion 401. A range farther from the projector 105 than the position corresponding to the distance 305 which corresponds to MPE 405, including the position corresponding to the distance 305, corresponds to the irradiation possible range. In this way, a range where the projection distance is sufficiently long and the radiation density of light is low is set to the irradiation possible range, so that the present invention can be applied. When decreasing the light irradiation density by using a diffuser panel, the irradiation possible range is obtained according to a relationship between the projection distance from the diffuser panel to the plate 111 and the irradiation density and the irradiation possible range is stored in the memory 120, so that the present invention can be applied. When the light emitting unit has an optical unit including a light collection unit such as a lens and a diffuser unit such as a diffuser panel, a portion of the optical unit nearest to the plate 111 which is the pressure unit is the light emitting end of the light emitting unit. The light emitting unit is used as a reference when the distance between the light emitting unit and the pressure unit is acquired. The distance between the light emitting end and the surface of the plate 111, which is the pressure unit, facing the subject is the projection distance and also the distance between the light emitting unit and the pressure unit. When the optical unit is not provided as shown in FIG. 5A, the light emitting portion 401 of the optical fiber is the light emitting end (the reference for acquiring the distance) of the light emitting unit.

Although the present embodiment has been described using an example in which there is one projector, even when there are a plurality of projectors, the present invention can be applied by obtaining the irradiation possible range on the basis of the characteristics of combined light from both sides. In other words, the present embodiment has been described using an example in which there is one Z coordinate that has a peak of the irradiation density as shown in FIG. 4B. However, it is possible to use an optical system where a plurality of coordinates have a peak.

In the present embodiment, a range in which the projection distance is greater than or equal to a certain threshold value is set to the irradiation possible range. However, it is possible to use an optical system in which the irradiation density increases at a distant position and set a range in which the projection distance is smaller than or equal to a certain threshold value to the irradiation possible range. Also, it is possible to have two threshold values and set a range in which the projection distance is between the two threshold values to the irradiation possible range.

The present embodiment has been described using an example in which an interval between a pair of plates (the distance between the plates) is measured by a potentiometer which is used as the position sensor 114. However, the method for measuring the position of the plate is not limited to this. For example, a sensor such as a photo-interrupter is provided at the position 304 at which the irradiation density of the light emitted from the light emitting unit is smaller than or equal to the MPE and it may be determined whether or not the plate is located nearer to the emitting end of the light emitting unit (the reference of light emission) than the position 304.

The present embodiment has been described using an example in which the holding mechanism transmits a rotational force of the handle to the plate 111 and moves the plate 111. However, the method for moving the plate 111 is not limited to this. For example, a foot switch is provided under a foot of the user and it is possible to use a holding mechanism in which a motor moves to move the plate 111 when the user presses the foot switch.

As described above, according to the first embodiment of the present invention, when capturing an image without reducing the light quantity emitted from the light emitting unit, the plate can be sufficiently away from the light emitting unit, so that it is possible to ensure both the contrast of the diagnostic image and the safety.

### Second Embodiment

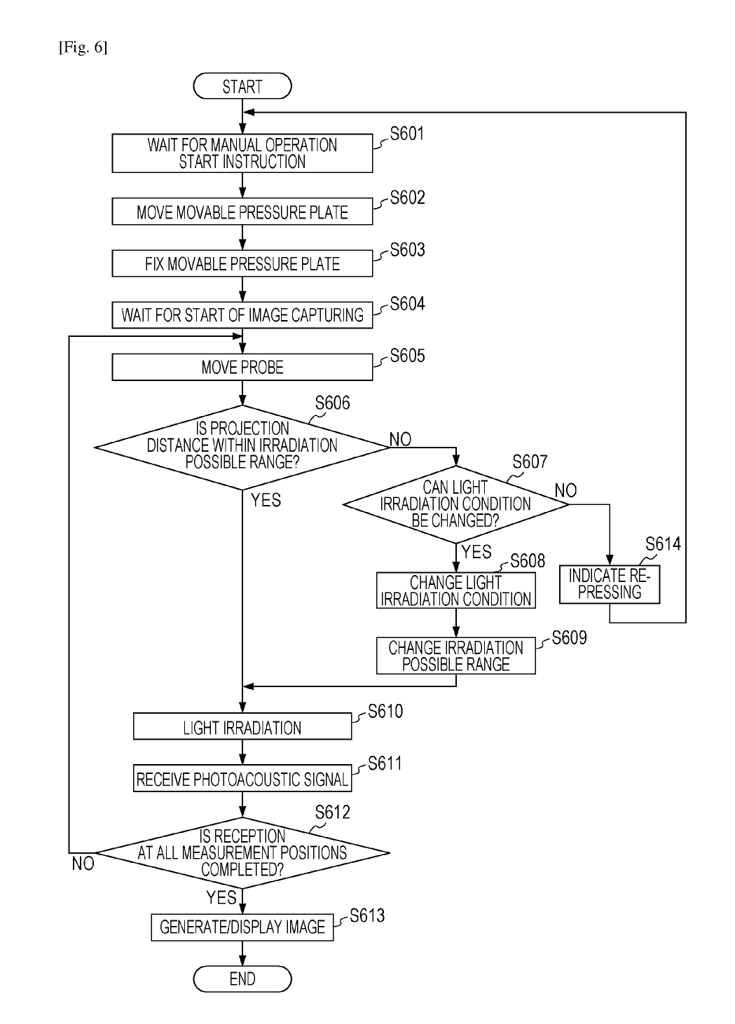

Next, a second embodiment of the present invention will be described. The difference of the second embodiment of the present invention from the first embodiment is that when the project distance is out of the irradiation possible range, the light source is controlled so that the irradiation density of the subject is smaller than or equal to the MPE. Since the block configuration diagram of the second embodiment is the same as that of the first embodiment, the second embodiment will be described with reference to FIG. 1. FIG. 6 is a flowchart showing an operation procedure of the photoacoustic apparatus of the present invention.

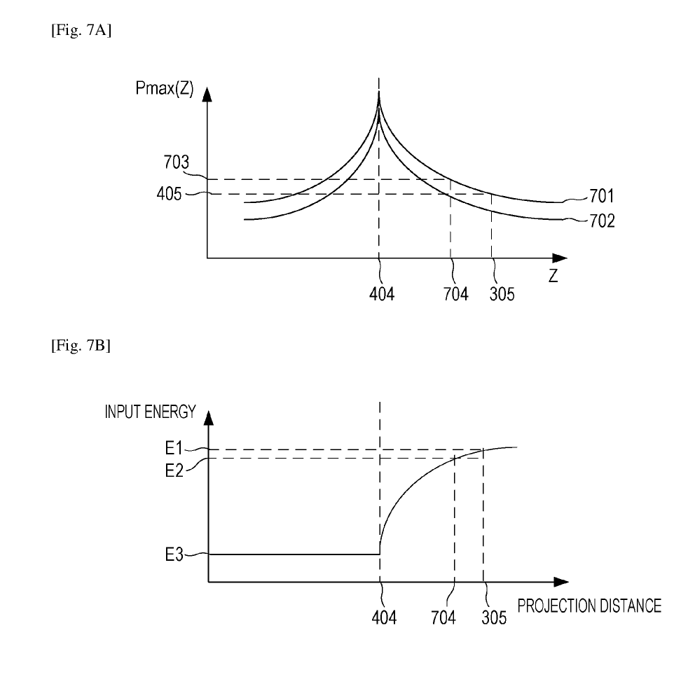

From step S601 to step S603, the subject 102 is pressed and fixed. These processes are the same as those of steps S201 to S203 in FIG. 2 described in the first embodiment. Subsequently, in steps S604 and step S605, the image capturing conditions are set and the probe is moved. These processes are the same as those of steps S205 to S206 in FIG. 2. Subsequently, in step S606, the CPU 118 reads the position of the plate 111 from the position sensor 114 and determines whether or not the projection distance 303 is within the irradiation possible range stored in the memory 120. If the projection distance 303 is out of the irradiation possible range, the CPU 118 proceeds to step S607 to reduce the light quantity emitted from the light source. In step S608, the CPU 118 reads an input energy corresponding to the projection distance from the memory 120 and determines whether or not the amount of energy to be inputted is greater than or equal to a predetermined value necessary to generate the photoacoustic wave. If the amount of energy is greater than or equal to the predetermined value, the CPU 118 proceeds to step S608. If the amount of energy is smaller than the predetermined value, the CPU 118 proceeds to step S614 and prompts the user to press the subject again in the same manner as in step S212 in FIG. 2. In the present embodiment, the amount of energy to be inputted corresponds to a charge voltage to cause the flash lamp of the light source to emit light. It is assumed that a table indicating a correspondence relationship between the projection distance and the amount of energy to be inputted is stored in the memory 120 in advance. The relationship between the amount of energy to be inputted and the projection distance will be described with reference to FIGS. 7A and 7B. FIG. 7A is a diagram in which the horizontal axis indicates the Z axis and the vertical axis indicates the peak value Pmax(z) of the irradiation density. Reference numeral 701 denotes a relationship between the Z coordinate and the peak value of the irradiation density when the amount of energy E1 to be inputted into the light source 103 is set. Reference numeral 702 denotes a relationship between the Z coordinate and the peak value of the irradiation density when an input energy E2 smaller than E1 is set to the light source 103. It is assumed that the initial input energy of the light source 103 is E1 and the initial irradiation possible range is a range in which the projection distance is greater than the distance 305. Reference numeral 405 denotes the MPE. According to the relationship 701, if the projection distance is out of the irradiation possible range when the input energy is E1, the irradiation density may exceed the MPE. For example, when the projection distance is a value indicated by the distance 704 in FIG. 7A, if the input energy is E1, the peak of the irradiation density is a value 703, which is greater than the MPE 405. On the other hand, as the input energy decreases, power of the pulsed light decreases accordingly and also the irradiation density decreases. When the input energy decreases to E2, the distance 704 is within the irradiation possible range. In this way, the irradiation possible range varies according to the input energy. FIG. 7B shows this situation. FIG. 7B is a diagram in which the horizontal axis indicates the projection distance and the vertical axis indicates the maximum input energy Et allowed in each projection distance. When the projection distance is greater than the distance 404 corresponding to the image forming position 404, if the maximum input energy Et is inputted into the light source 103, the irradiation density at the projection distance equals to the MPE.

As shown in FIG. 7B, Et is equal to E1 at the distance 305 and Et is equal to E2 at the distance 704. In the optical system in FIG. 5A, Et becomes the minimum value E3 at the image forming position. When the projection distance is smaller than or equal to the distance corresponding to the image forming position, Et is E3. This is to eliminate the possibility that the irradiation density exceeds the MPE in the subject.

In the present embodiment, the relationship between the projection distance and the allowed input energy shown in FIG. 7B is measured in advance and stored in a table in the memory 120. Subsequently, in step S608, the CPU 118 issues an instruction to the light source drive unit 119 and changes the setting of the input energy. Subsequently, in step S609, the CPU 118 updates the irradiation possible range in the memory 120 to a range where the projection distance is greater than the distance 704. Thereafter, when the projection distance changes to a distance out of the irradiation possible range while an image is being captured, the input energy of the light source 103 is changed again. Subsequently, in step S610, the CPU 118 causes the light source 103 to emit the pulsed light in the same manner as in step S208. If the input energy is changed in step S608, the pulsed light is generated by using the updated input energy and the pulsed light is irradiated to the subject 102 through the optical system of the projector 105. Thereby, even when the thickness of the subject is greater than estimated and the projection distance is small, the peak of the irradiation density of the light irradiated to the subject does not exceed the MPE shown in FIG. 7A. Subsequently, in steps S611 to S613, the same processes as those of step S209 to S211 in the first embodiment of the present invention are performed, so that an image of the acquired photoacoustic signals is formed and displayed.

In the present embodiment, in the change of the light irradiation condition in step S607, the emitted light quantity is controlled by changing the amount of energy inputted into the light source 103. However, the method of controlling the light source is not limited to this. For example, the irradiation condition may be changed by changing the excitation time or controlling the frequency of the pulsed light. When the thickness of the subject is large and the S/N ratio largely decreases if the emitted light quantity is reduced, it is possible to change not only the light quantity emitted from the light source, but also the number of times when the photoacoustic signal is acquired, which is set in step in step S604. For example, instead of reducing the light quantity emitted from the light source, by increasing the number of times when the photoacoustic signal is acquired, it is possible to reduce the decrease of the S/N ratio of the photoacoustic signal. In the present embodiment, an example is described in which the input energy is limited when the projection distance is out of the irradiation possible range. However, the input energy shown in FIG. 7B may be set according to the projection distance. In this case, when the projection distance becomes the distance 704, the input energy decreases from E1 to E2, and thereafter when the projection distance returns to the distance 305, the input energy increases from E2 to E1. Even every time the projection distance is changed by a retry of the manual operation or the like, it is possible to emit the maximum amount of light that can be irradiated and obtain a diagnostic image with high contrast.

As described above, according to the second embodiment of the present invention, even when the thickness of the subject is large and the projection distance is small, it is possible to capture an image without retrying the pressure fixation while the safety of the subject is ensured by changing the light irradiation condition within an allowable range.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-092274, filed Apr. 13, 2012, which is hereby incorporated by reference herein in its entirety.

### REFERENCE SIGNS LIST

102 Subject
103 Light source
104 Optical fiber
105 Projector
108 Photoacoustic wave
109 Probe
110 Plate
111 Plate
114 Position sensor
117 Display
118 CPU
119 Light source drive unit

1. A subject information acquisition apparatus for acquiring characteristic information of a subject by receiving a photoacoustic wave generated by irradiating the subject with light, the subject information acquisition apparatus comprising:
a pressure unit configured to press the subject;
a light emitting unit configured to emit light to the subject through the pressure unit;
a distance information acquisition unit configured to acquire information related to a distance between the pressure unit and the light emitting unit; and
a control unit configured to control an operation of the light emitting unit on the basis of the information related to the distance acquired by the distance information acquisition unit.

2. The subject information acquisition apparatus according to claim 1, wherein the control unit controls emission of light of the light emitting unit on the basis of the information related to the distance.

3. The subject information acquisition apparatus according to claim 2, wherein the control of emission of light is a control of an emitted light quantity.

4. The subject information acquisition apparatus according to claim 3, wherein the control of emission of light is to prevent the light from being emitted.

5. The subject information acquisition apparatus according to claim 3, wherein the control of the emitted light quantity is performed by a control of an amount of energy inputted into the light emitting unit.

6. The subject information acquisition apparatus according to claim 3, wherein the control of the emitted light quantity is performed by a control of excitation time of the light emitting unit.

7. The subject information acquisition apparatus according to claim 1, further comprising: a presentation unit configured to present the information related to the distance.

8. The subject information acquisition apparatus according to claim 7, wherein the presentation unit is a display unit.

9. The subject information acquisition apparatus according to claim 7, wherein the presentation unit is a voice generation unit.

10. The subject information acquisition apparatus according to claim 1,
wherein the pressure unit includes a pair of plates facing each other which press the subject when one plate moves while the other plate is fixed, and
the light emitting unit emits light to the subject through the one plate of the pair of plates.

11. The subject information acquisition apparatus according to claim 10, wherein the distance information acquisition unit acquires the information related to the distance between the pressure unit and the light emitting unit by measuring a position of the one plate of the pair of plates.

12. The subject information acquisition apparatus according to claim 10, wherein the distance information acquisition unit acquires the information related to the distance between the pressure unit and the light emitting unit by measuring an interval between the pair of plates.

13. A method for acquiring characteristic information of a subject by receiving a photoacoustic wave generated when a light emitting unit emits light to the subject pressed by a pressure unit through the pressure unit, the method comprising:
a step of pressing the subject by the pressure unit;
a step of acquiring information related to a distance between the pressure unit and the light emitting unit; and
a step of controlling an operation of the light emitting unit on the basis of the acquired information related to the distance.

14. The method for acquiring characteristic information of a subject according to claim 13, further comprising: a step of presenting the acquired information related to the distance.

15. The method for acquiring characteristic information of a subject according to claim 14, wherein the step of presenting the acquired information is performed by displaying the acquired information.

16. The method for acquiring characteristic information of a subject according to claim 14, wherein the step of presenting the acquired information is performed by a voice.

17. The method for acquiring characteristic information of a subject according to claim 14, wherein the information related to the distance is information that prompts to increase the distance.
